(19) United States
(12) Patent Application Publication
(10) Pub. No.: US 2014/0256051 A1
(43) Pub. Date: Sep. 11, 2014

(54) DECARBOXYLATION DOSIMETRY

(71) Applicant: University of Rochester, Rochester, NY (US)

(72) Inventors: William A. Bernhard, Honeoye Falls, NY (US); Richard M. Watson, Rochester, NY (US); Paul Black, Rochester, NY (US)

(73) Assignee: University of Rochester, Rochester, NY (US)

(21) Appl. No.: 14/199,948

(22) Filed: Mar. 6, 2014

Related U.S. Application Data
(60) Provisional application No. 61/773,758, filed on Mar. 6, 2013.

(51) Int. Cl. G01N 33/72; G01N 33/68

(52) U.S. Cl. CPC G01N 33/721; G01N 33/6803
USPC 436/66; 436/90; 422/68.1

(57) ABSTRACT

Methods and apparatuses of the present disclosure provide accurate and precise radiation dosimetry based on decarboxylation of a reactant upon exposure to ionizing radiation. The decarboxylation reaction yields carbon dioxide and stable end products, which can be detected and measured, and correlated to determine an amount of radiation exposure. The reactants include carboxyl acids, such as amino acids and others occurring naturally in mammals. The dosimetry may be performed in real-time or retrospectively by assaying a sample of tissue from a patient. Also disclosed is a device which detects and measures radiation dosages based on decarboxylation of a reactant contained therein.

### RELATED APPLICATIONS

This application claims the benefit of U.S. Provisional Application No. 61/773,758, filed Mar. 6, 2013.

### GOVERNMENT LICENSE RIGHTS

This invention was made with government support under 2-R01-CA32546 awarded by the National Institutes of Health and the National Cancer Institute. The Government has certain rights to this invention.

### FIELD

The present disclosure related to the field of radiation dosimetry, including related methods and apparatuses.

### BACKGROUND

Radiation dosimetry is the measurement and calculation of the absorbed dose in matter and tissue resulting from the exposure to indirect and direct ionizing radiation (e.g., X-rays, radioactivity). Absorbed dose (also known as total ionizing dose or “TID”) is a measure of the energy deposited in a medium by ionizing radiation per unit mass. Absorbed dose is measured in units of gray (Gy). When ionizing radiation is used to treat cancer, the doctor will usually prescribe the radiotherapy treatment in Gy. When risk from ionizing radiation is being discussed, a related unit for equivalent dose, the sievert (Sv) is be used.

Absorbed dose is distinguishable from other, more easily characterized properties of a radiation source (such as the energy of emitted photons or the radioactive activity of a source). Exposure to ionizing radiation will give a dose which is dependent on the activity, time of exposure, energy of the radiation emitted, distance from the source, and shielding. Due to the number of factors that affect absorbed dose, it is directly measured by analyzing the specific material or tissue which received the dose. Thereby, chemical products produced from the absorption of ionizing radiation are quantified.

In general the chemical yield of damage products from absorbed radiation dose is susceptible to dose saturation once a high enough dose is reached. Dose saturation is the result of radiation repair that occurs when a hole is destroyed by addition of an excess electron from a subsequent track of radiation, or when a damage product is converted back into the parent molecule by absorption of additional radiation. In both of these cases, the dose response deviates from linearity and reaches a plateau, which is described as dose saturation.

### SUMMARY

The methods and devices of the present disclosure may use naturally occurring and ubiquitous amino acids in biodosimetry. This invention presents a powerful method of assessing radiation dose to a person or population exposed to an unknown amount of radiation, and may be used in a rapid, high-throughput system. By using the decarboxylation dosimetry methods disclosed, specific proteins may be extracted and analyzed to assess the dose and biological effect of a population exposed to radiation in a nuclear accident or other medical, industrial, or security-related radiation incident.

One aspect of the present disclosure is that the absorbed dose can be directly measured from the irradiated tissue. This may be used in radiation therapy to provide a measurement of the actual dose of radiation a patient has received in a specific organ or region of their body. Additionally, irradiation of hemoglobin protein under physiological conditions, followed by standard protein hydrolysis and analysis by LCMS demonstrates the ability of decarboxylation dosimetry to be useful as a biodosimeter.

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 55. The other clauses can be presented in a similar manner.

1. A method of determining an effect of ionizing radiation, comprising:

allowing a sample comprising a carboxylic acid to be exposed to an initially unknown amount of ionizing radiation, such that a single-electron oxidized product and carbon dioxide are produced from the carboxylic acid;

exposing the single-electron oxidized product to a radioprotectant, such that the single-electron oxidized product form an end product;

determining an amount of the end product; and

based on the amount of the end product, determining the amount of ionizing radiation.

2. The method of clause 1, wherein the sample comprises one or more of an amino acid, a dicarboxylic acid, a fatty acid, a sugar acid, a hydroxy acid, an acetic acid, a keto acid, a bile acid, a propionic acid, an aromatic acid, a carboxylic acid derivative, naphthoic acid, a nicotinic acid, a tricarboxylic acid, and a glucuronic acid.

3. The method of clause 1, wherein the sample comprises a beta amino acid.

4. The method of clause 1, wherein the sample comprises beta-alanine and the end product is ethylamine.

5. The method of clause 1, wherein the sample comprises gamma-aminobutyric acid and the end product is propylamine.

6. The method of clause 1, wherein the sample comprises glutamic acid and the end product is 4-aminobutyric acid and 2-aminobutyric acid.

7. The method of clause 1, wherein the radioprotectant comprises sulfur.

8. The method of clause 1, wherein the radioprotectant is ethanethiol.

9. The method of clause 1, whereby the determining is performed by one or more of gas chromatography, liquid chromatography, mass spectrometry, NMR spectroscopy, infrared spectroscopy.

10. A method of retrospectively determining an effect of ionizing radiation, comprising:

assaying a sample of tissue from a patient that has been exposed to ionizing radiation;

measuring an amount of a radiation product in the tissue, wherein the radiation product is a product of decarboxylation of an amino acid in the tissue in response to the ionizing radiation;

correlating the amount of the radiation product to an amount of ionizing radiation absorbed by the patient.

11. The method of clause 10, wherein measuring comprises extracting and hydrolyzing hemoglobin to produce a product.

12. The method of clause 11, wherein measuring further comprises analyzing the product by liquid chromatography coupled mass spectrometry

13. The method of clause 10, wherein the amino acid is beta-alanine and the radiation product is ethylamine.

14. The method of clause 10, wherein the amino acid is glutamic acid and the radiation product is alpha-aminobutyric acid.

15. The method of clause 10, wherein the tissue is blood.

16. The method of clause 10, wherein the ionizing radiation is provided as radiotherapy.

17. The method of clause 10, wherein the amount of ionizing radiation is between about 10 kGy and about 500 kGy.

18. A real-time dosimetry device, comprising:

a first chamber containing a reactant comprising a carboxylic acid configured to undergo loss of carbon dioxide to form an analyte, comprising a single-electron oxidized product, upon absorption of ionizing radiation;

a second chamber containing a detection reagent configured to emit a response in the presence of the analyte; and

a gas-permeable membrane separating the first chamber from the second chamber and configured to permit diffusion of the analyte.

19. The real-time dosimetry device of clause 18, wherein the second chamber contains a detection medium configured to emit a response in the presence of carbon dioxide.

20. The real-time dosimetry device of clause 18, wherein the reactant is beta-alanine and the analyte comprises ethylamine.

Additional features and advantages of the subject technology will be set forth in the description below, and in part will be apparent from the description, or may be learned by practice of the subject technology. The advantages of the subject technology will be realized and attained by the structure particularly pointed out in the written description and claims hereof as well as the appended drawings.

It is to be understood that both the foregoing general description and the following detailed description are exemplary and explanatory and are intended to provide further explanation of the subject technology as claimed.

### BRIEF DESCRIPTION OF THE DRAWINGS

The accompanying drawings, which are included to provide further understanding of the subject technology and are incorporated in and constitute a part of this specification, illustrate aspects of the subject technology and together with the description serve to explain the principles of the subject technology.

FIG. 1 shows an exemplary reaction of beta-alanine following ionization (resulting from absorbed radiation dose), according to some embodiments of the present disclosure.

FIG. 2 shows an exemplary reaction of 4-aminobutyrate (aka gamma-aminobutyric acid) following ionization, according to some embodiments of the present disclosure.

FIG. 3 shows an exemplary reaction of glutamic acid following ionization, according to some embodiments of the present disclosure.

FIG. 4 shows an exemplary NMR Spectra of 4-aminobutyrate (top), propylamine (middle), and aminopropanol (bottom), according to some embodiments of the present disclosure.

FIG. 5 shows an exemplary NMR Spectra of 4-aminobutyrate following 10 kGy, 50 kGy, 500 kGy doses, according to some embodiments of the present disclosure.

FIG. 6 shows an exemplary NMR Spectra of glutamate mixed with alpha-aminobutyric acid (bottom), alpha-aminobutyric acid (second from bottom), and glutamate following 0, 3 kGy, 10 kGy, 30 kGy and 100 kGy doses (top 5 spectra), according to some embodiments of the present disclosure.

FIGS. 7A and 7B show an exemplary dose response of beta-alanine (top) and glutamate (bottom) as measured by integration of product peaks in NMR corresponding to ethylamine and alpha-aminobutyric acid, respectively. The x-axis is dose (kGy) and the y-axis is normalized NMR peak area (unitless), according to some embodiments of the present disclosure.

FIG. 8 shows an exemplary dose response of glutamate as measured by LCMS analysis and quantification of alpha-aminobutyric acid, according to some embodiments of the present disclosure.

FIG. 9 shows an exemplary real-time dosimetry device, according to some embodiments of the present disclosure.

### DETAILED DESCRIPTION

In the following detailed description, numerous specific details are set forth to provide a full understanding of the subject technology. It will be apparent, however, to one ordinarily skilled in the art that the subject technology may be practiced without some of these specific details. In other instances, well-known structures and techniques have not been shown in detail so as not to obscure the subject technology.

Decarboxylation dosimetry is a process in which certain amino acids undergo well-defined radiation chemistry when ionized by radiation and yield specific, detectable products related to the absorbed dose over a broad range of doses. The properties of this process allow these amino acids and related chemical structures to be used as general purpose radiation dosimeters or biodosimeters. The dose response is substantially linear over several orders of magnitude. The dose response provides accurate dose information over a broad range of dosages.

Decarboxylation products of amino acids and similar molecules are not easily susceptible to dose saturation. Therefore, they are excellent candidates for dosimeters in a broad range of fields, from extremely low-dose (e.g., mGy range) detection (e.g., in biological tissue and environmental radiation risk assessment) to measuring medium to high doses (e.g., Gy to kGy range) used in industrial processes or to assess nuclear accidents or national security concerns. This method may also be employed to measure accurately the extremely high doses used in high energy x-ray crystallography (e.g., MGy range).

The broad applicability, as well as simplicity of this method should make it an extremely attractive commercial tool in a vast number of health, scientific, government, and industrial fields.

Ionizing radiation interacts with matter by producing radical intermediates which are the result of either the addition of an excess electron (“EE”) or the production of a “hole” which is the loss of an electron. Each ionization results in exactly one EE and one hole. Therefore the absorbed dose is directly related to yield of chemical products resulting from the presence of either an EE or a hole.

According to some embodiments, a method of determining an effect of ionizing radiation by decarboxylation dosimetry is disclosed herein. According to some embodiments, a method of decarboxylation dosimetry may comprise allowing a reactant comprising a carboxylic acid to be exposed to ionizing radiation, whereby a precursor carboxyl radical cation (e.g., a single-electron oxidized product) and carbon dioxide are produced. According to some embodiments, ionizing radiation results in decarboxylation of a compound comprising at least one carboxyl group. Exemplary reactants may include carboxylic acids. Examples of reactants include amino acids, dicarboxylic acids, fatty acids, sugar acids, hydroxy acids, acetic acids, keto acids, bile acids, propionic acids, aromatic acids, carboxylic acid derivatives, naphthoic acids, nicotinic acids, tricarboxylic acids, and glucuronic acids. Examples of reactants further include compounds occurring naturally in animals, including mammals.

According to some embodiments, each and every single unit of absorbed radiation dose (i.e., each photon) by a reactant, such as an amino acid, results in exactly one molecule of carbon dioxide and one molecule of a stable radiation product due to initial radical cation formation, such as a primary amine. Moreover, because carbon dioxide is lost from the system, the linear and directly-related dose response occurs over a broad range of doses and dose rates.

According to some embodiments, a method of decarboxylation dosimetry may comprise exposing the site of radical cation production, produced by one-electron oxidation, to a radioprotectant. One or more radioprotectants may be used. Examples of radioprotectants include ethanethiol, amifostine, sodium diethyldithiocarbamate, adeturon, aminothiol, fosfomycin, sodium thiosulfate, and thiolactone. A radioprotectant may be configured to sequester and/or recycle oxidative damage. For example, a radioprotectant may comprise sulfur. In the presence of a radioprotectant, the radical cation forms a stable end-product, which can be detected and quantified by one or more processes.

According to some embodiments, a method of decarboxylation dosimetry may comprise determining an amount of the end product. Examples of mechanisms and methods for making such a determination include use of gas chromatography, liquid chromatography, mass spectrometry, NMR spectroscopy, infrared spectroscopy, gas chromatography-mass spectrometry, liquid chromatography-mass spectrometry, gas chromatography-infrared spectroscopy, liquid chromatography-NMR spectroscopy as well as other methods of substance identification, and combinations thereof. According to some embodiments, an amount of an end product produced by absorption of ionizing radiation may be correlated with an amount an absorbed dose, as discussed further herein.

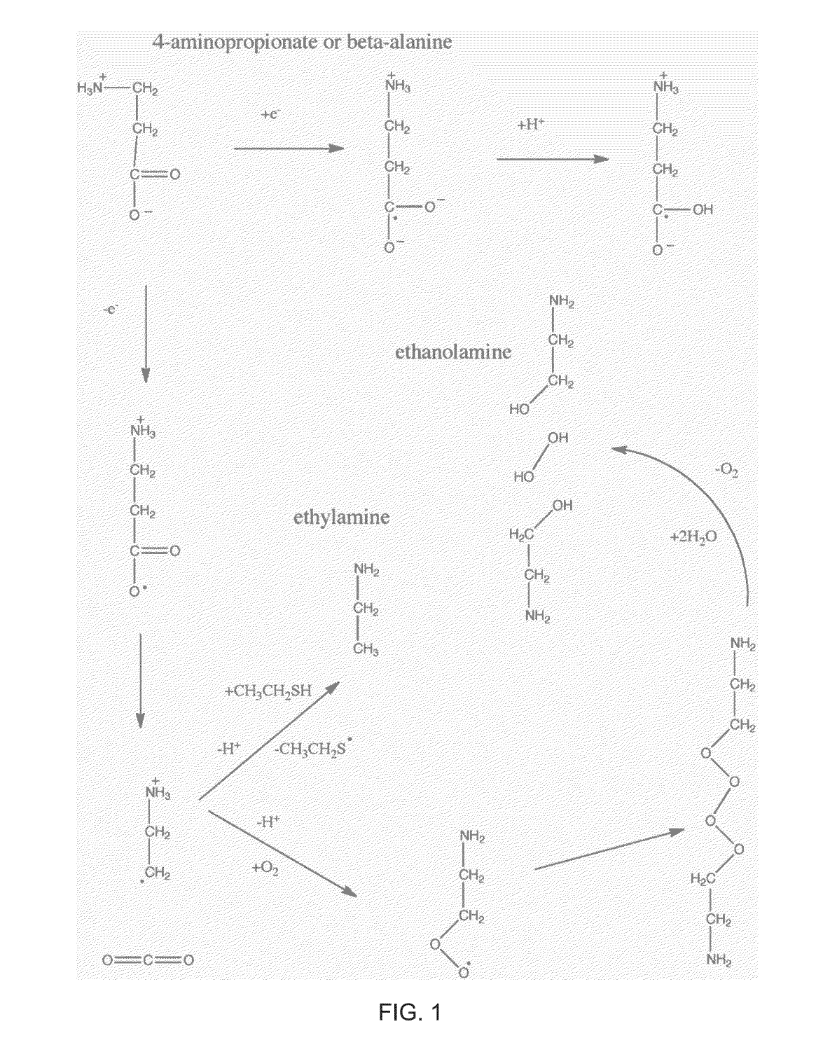

According to some embodiments, an exemplary method of decarboxylation dosimetry may include a reaction of beta-alanine. Beta alanine is a small, naturally occurring beta amino acid, reactive to ionizing radiation. As shown in FIG. 1, the production of a hole on beta-alanine produces a carboxyl radical cation, which subsequently undergoes loss of carbon dioxide to form ethylamine. In the presence of ethanethiol or a similar radioprotectant, this radical goes on to form the stable end-product, which can easily be detected and quantified by gas chromatography or gas chromatography-mass spectrometry.

FIG. 1 shows an exemplary reaction of beta-alanine following ionization (resulting from absorbed radiation dose), according to some embodiments of the present disclosure. In FIGS. 1-3, +e—indicates the addition of an excess electron (EE), whereas −e—indicates the loss of an electron to produce a hole. Either path can be used for quantification because each ionization results in exactly one EE and one hole. Decarboxylation dosimetry makes use of the chemical reaction resulting from the “hole”, which is the downward pathway on the left in this figure. As shown, formation of a hole on beta-alanine leads to loss of CO₂ and eventual formation of the stable end-product ethylamine, a volatile primary amine with a by of 16° C.

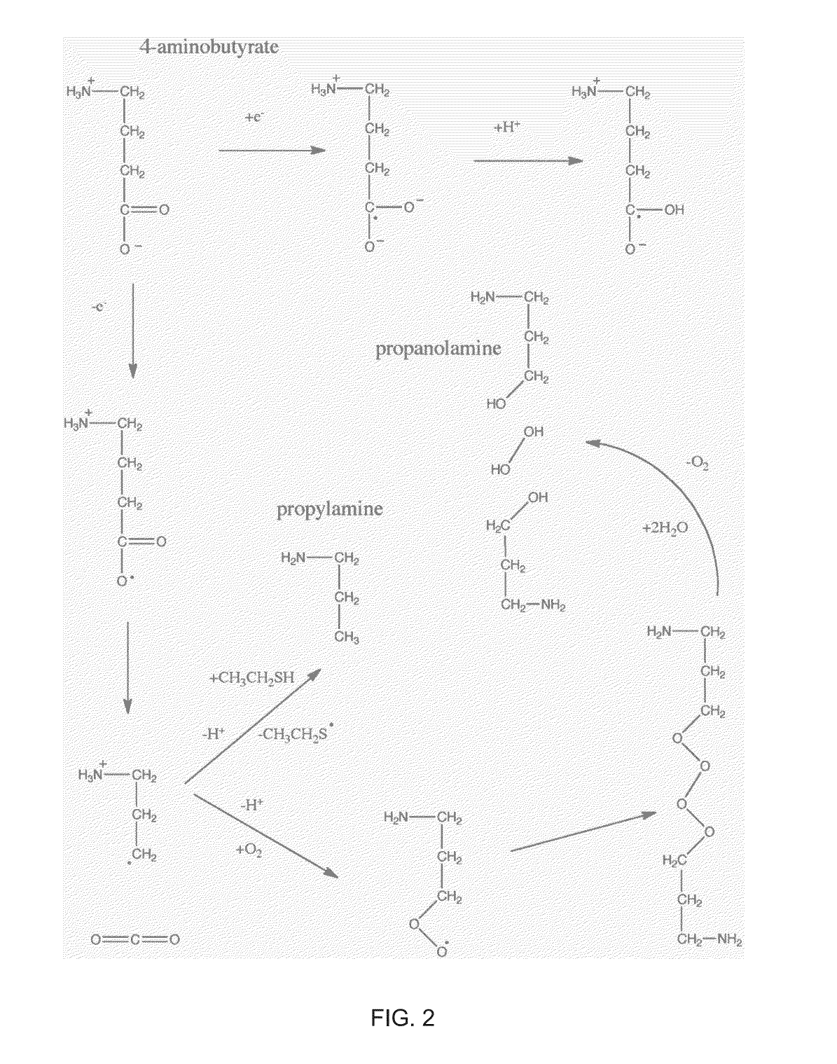

FIG. 2 shows an exemplary reaction of 4-aminobutyrate (aka gamma-aminobutyric acid) following ionization, according to some embodiments of the present disclosure. As the downward pathway on the left shows, formation of a “hole” on the parent molecule leads to loss of CO₂ and eventual formation of the stable end product propylamine, a primary amine with a by of 48° C.

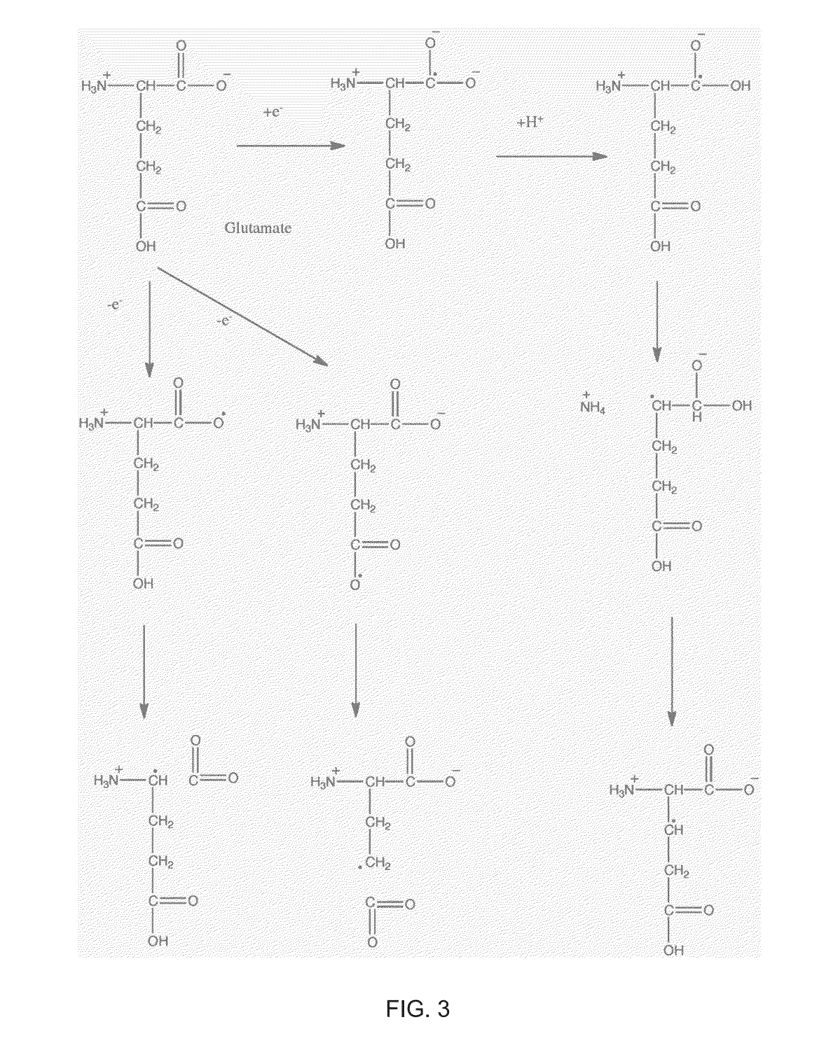

FIG. 3 shows an exemplary reaction of glutamic acid following ionization, according to some embodiments of the present disclosure. As the downward pathway on the left shows, formation of a “hole” on glutamate leads to loss of CO2 and eventual formation of the two stable end-products 4-aminobutyric acid (bottom left) and 2-aminobutyric acid (bottom right). When glutamic acid is incorporated in a protein, the left-most pathway is unavailable, and only 2-aminobutyric acid is produced. In either case, the products can be quantified by LCMS and other analytical techniques.

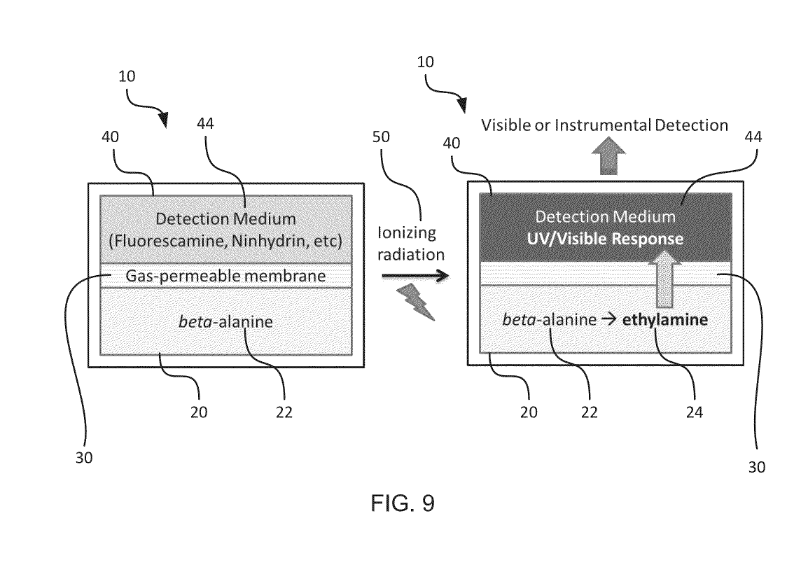

According to some embodiments, a real-time dosimetry device is disclosed herein. According to some embodiments, as shown in FIG. 9, a real-time dosimetry device 10 comprises a reactant chamber 20 containing a reactant 22 comprising a carboxylic acid configured to undergo loss of carbon dioxide to form an analyte 24. The analyte 24 may comprise a precursor radical cation or a radiation product. The reactant 22 may be any one or more of the compounds disclosed herein. For example, the reactant 22 may be any carboxyl-containing compound capable of undergoing decarboxylation upon absorption of ionizing radiation. The reactant may include carboxylic acids, such as amino acids, dicarboxylic acids, fatty acids, sugar acids, hydroxy acids, acetic acids, keto acids, bile acids, propionic acids, aromatic acids, carboxylic acid derivatives, naphthoic acids, nicotinic acids, tricarboxylic acids, and glucuronic acids. The analyte 24 may comprise a product of decarboxylation. For example, a single-electron oxidized product, precursor radical cation, or radiation product (e.g., ethylamine) may be produced by decarboxylation of a product (e.g., beta-alanine).

According to some embodiments, as shown in FIG. 9, a real-time dosimetry device 10 comprises a membrane 30 separating the reactant chamber 20 from the detection chamber 40. The membrane 30 may be gas-permeable. The membrane 30 may be selectively permeable. For example, the membrane 30 may be configured to permit diffusion of the analyte 24 from the reactant chamber 20 to the detection chamber 40. The membrane 30 may be configured to retain the reactant 22 or other materials of a certain character (size, etc.).

According to some embodiments, as shown in FIG. 9, a real-time dosimetry device 10 comprises a detection chamber 40 containing detection medium 44 configured to emit a response in the presence of an analyte 24. The detection medium 44 may include any material that is capable of providing a detectable output in response to its reaction with an analyte 24. The output may be measurable by a user or another device. For example, the detection medium 44 may provide a visual output in response to reaction with an analyte 24, alerting a user of absorption of ionizing radiation 50. The detection medium could be any number of common reagents that react with primary amines to produce a detectable response. For example fluorescamine or ninhydrin, both of which would produce a visible, measurable response for each molecule of ethylamine or other analyte 24 that evolves from the reactant chamber 20 after radiation exposure. A detection medium may be produced linearly as a function of an absorbed dose and be detectable within dose ranges of interest.

According to some embodiments, a carbon dioxide detection system could be used in place of or in addition to a system for detecting an analyte 24. For example, the detection chamber 40 may comprise a device or substance for detecting an amount of carbon dioxide emitted by decarboxylation upon absorption of ionizing radiation 50. The amount of carbon dioxide emitted may be correlated with an amount of radiation absorbed based on analysis of the processes disclosed herein.

According to some embodiments, as shown in FIG. 9, a method of using a real-time dosimetry device 10 is disclosed herein. Upon absorption of ionizing radiation 50, at least a portion of a reactant 22 in the reactant chamber 20 undergoes decarboxylation to produce an analyte 24. The analyte 24 may be formed by creation of a precursor radical cation or single-electron oxidized product, followed immediately by conversion to a stable end product. The membrane 30 allows the analyte 24 to cross from the reactant chamber 20 to the detection chamber 40. In the presence of the analyte 24, the detection medium 44 produces a detectable output. A device or a user may sense the output to determine a level of ionizing radiation 50 that has been absorbed.

According to some embodiments, a real-time dosimetry device 10 may be used in applications for which real-time radiation exposure alerting or measurement is desired. For example, personnel could wear a button-sized device that would alert them when entering an environment where radiation (e.g., radioactivity or high-energy electromagnetic radiation) is present. Industrial, medical, and government radiation workers would also benefit from low-cost, accurate, and rapid detection that such a device would provide.

The mechanism described for reactants above also applies to reactants that are ubiquitous in biological systems. For example, reactant sensitive to ionizing radiation occur naturally in plants and animals, including mammals and humans. Retrospective biodosimetry according to the present disclosure provides a method of assessing radiation dose to a person or population exposed to an initially unknown amount of radiation. By using the decarboxylation dosimetry method described herein, specific proteins may be extracted from tissues and analyzed to assess quickly the dose and biological effect of a population exposed to radiation in a nuclear accident or other medical, industrial, or security-related radiation incident.

According to some embodiments, a method of retrospectively determining an effect of ionizing radiation is disclosed herein. A sample of tissue (e.g., blood) from a patient that has been exposed to ionizing radiation may be assayed. The amount of a radical cation, single-electron oxidized product, or radiation product in the tissue may be measured. For example, hemoglobin may be extracted and hydrolyzed to produce a product that may be detected. The product may be analyzed by substance analysis methods, such as liquid chromatography coupled mass spectrometry.

An assay may be performed as an investigative (analytic) procedure for qualitatively assessing or quantitatively measuring the presence or amount or the functional activity of a target entity (the analyte) which can be a drug or biochemical substance or a cell in an organism or organic sample. “Analyte” refers to the measured entity of the assay. The assay may measure an intensive property of the analyte and express it in the relevant measurement unit (e.g. molarity of a reactant, dosage of radiation absorbed, etc.).

The assay may include or be preceded by preanalytic steps, such as information communication (e.g. request to perform an assay and further information processing) or specimen handling (e.g. collection transport and processing). The assay may include or be followed by post-analytic steps, such as documentation, verification, and transmission of results.

According to some embodiments, as assay procedure may include sample processing/manipulation step to present a target in a discernible/measurable form to a discrimination/identification/detection system. Sample processing may involve, for example, a centrifugal separation, washing, filtration, or capture by some form of selective binding. Sample processing may involve modifying the target (e.g., cutting down the target into pieces, for example in mass spectrometry).

According to some embodiments, as assay procedure may include a target specific discrimination/identification step to discriminate from background (noise) of similar components and specifically identify a particular target component in a biological material by its specific attributes.

According to some embodiments, as assay procedure may include a signal (or target) amplification step. The presence and quantity of an analyte is converted into a detectable signal generally involving some method of signal amplification, so that it can be easily discriminated from noise and measured.

According to some embodiments, as assay procedure may include a signal detection (and interpretation) step. The amplified signal may be deciphered into an interpretable output that can be quantitative or qualitative. It can be visual or manual very crude methods or can be vary sophisticated electronic digital or analog detectors.

According to some embodiments, as assay procedure may include a signal enhancement and noise filtering step. Such a step may be done at any or all of the steps above. Since the more downstream a step/process during an assay, the higher the chance of carrying over noise from the previous process and amplifying it, multiple steps in a sophisticated assay might involve various means of signal-specific sharpening/enhancement arrangements and noise reduction or filtering arrangements. These may simply be in the form of a narrow band-pass optical filer, or a blocking reagent in a binding reaction that prevents nonspecific binding or a quenching reagent in a fluorescence detection system that prevents “autofluorescence” of background objects.

According to some embodiments, as assay procedure may include nuclear magnetic resonance spectroscopy (“NMR spectroscopy”), as disclosed herein. NMR spectroscopy is a research technique that exploits the magnetic properties of certain atomic nuclei to determine physical and chemical properties of atoms or the molecules in which they are contained. NMR spectroscopy provides detailed information about the structure, dynamics, reaction state, and chemical environment of molecules.

According to some embodiments, as assay procedure may include liquid chromatography-mass spectrometry (“LCMS”). Liquid chromatography-mass spectrometry is a chemistry technique that combines the physical separation capabilities of liquid chromatography (or HPLC) with the mass analysis capabilities of mass spectrometry. Liquid chromatography-mass spectrometry has very high sensitivity and selectivity. Generally its application is oriented towards the general detection and potential identification of chemicals in the presence of other chemicals (in a complex mixture).

According to some embodiments, an amount of a radiation product is indicative of decarboxylation of an amino acid or other organic carboxylic acid that is responsive to the ionizing radiation. The amount may be or represent an absolute amount, a relative amount, a change in an amount, or a ratio. For example, the reactant chosen may have a known or expected amount in the sample tissue. Accordingly, the amount of the radical cation, single-electron oxidized product, or radiation product in relation to the known or expected amount of the reactant may be correlated with an amount of ionizing radiation absorbed by the patient. For example, two or more assays may be performed, each at different times, wherein the difference across the two or more assays represents the exposure to ionizing radiation in the time span between the two or more assays. Any two results may be compared in this manner.

For example, glutamic acid proceeds through a mechanism similar to the one outlined above to produce CO2 and alpha-aminobutyric acid. Human hemoglobin contains a specific number of glutamic acid residues. By taking a drop of blood from a patient, hemoglobin can be extracted and hydrolyzed by standard methods. The resulting sample can be analyzed by liquid chromatography coupled mass spectrometry to quantify accurately the amount of alpha-aminobutyric acid present, which can be directly related to the absorbed radiation dose. This presents a high-throughput method to assess radiation dose quickly.

According to some embodiments, a hemoglobin analysis described above may be used or adapted to assess a delivered radiation dose in a case of radiotherapy (e.g., for cancer treatment). One aspect of this method is that the absorbed dose can be directly measured from the irradiated tissue. In the absence of blood in any given biological tissue, other abundant and highly-conserved proteins containing glutamic acid (or another carboxylic acid-containing amino acid such as aspartate) may be measured and correlated with an amount of a dosage received.

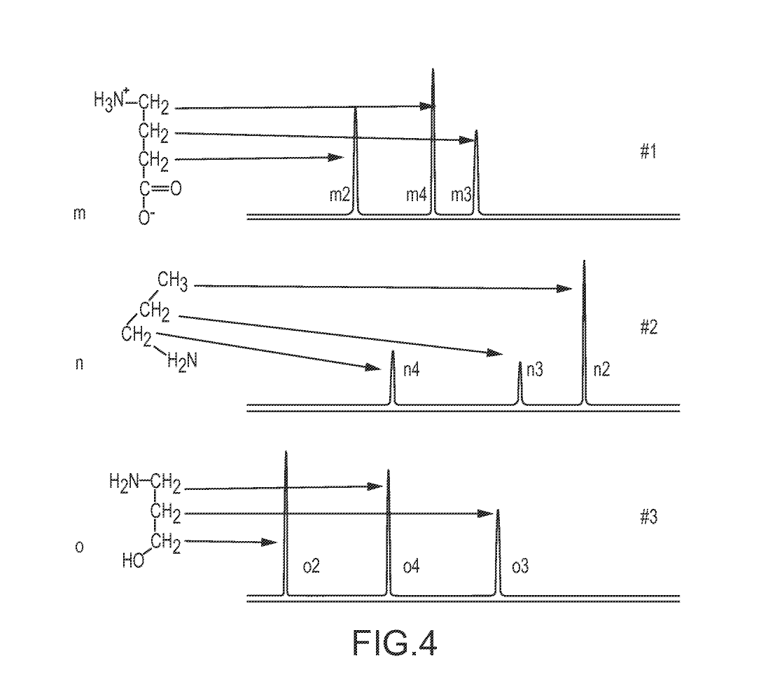

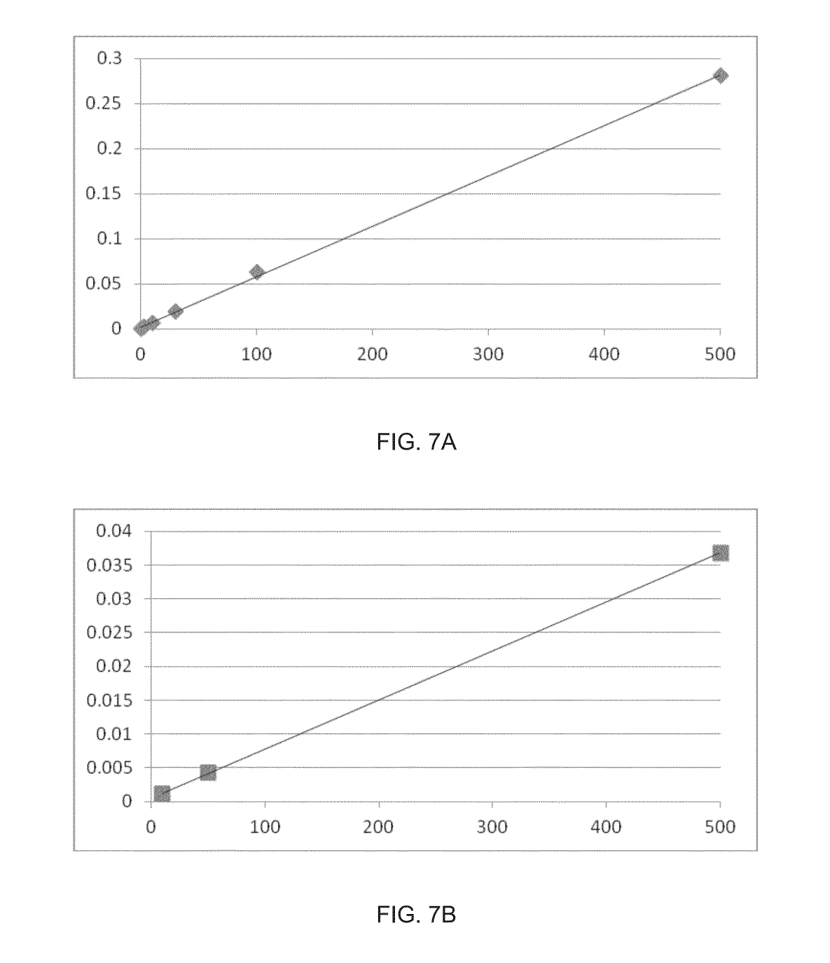

According to some embodiments, dose-response curves are obtainable from NMR, to demonstrate the dose-dependent production of a radical cation, single-electron oxidized product, or radiation product from a reactant by decarboxylation. For example, as disclosed herein, the NMR Spectra of ethylamine from beta-alanine, propylamine from gamma-aminobutyric acid, and alpha-aminobutyric acid from glutamic acid (FIGS. 4-6) are shown. As shown in FIGS. 7A and 7B, in all cases, the chemical yield of radiation products is substantially linear with respect to dose over the range tested (10 kGy to 500 kGy).

FIG. 4 shows an exemplary NMR Spectra of 4-aminobutyrate (top), propylamin (middle), and aminopropanol (bottom), according to some embodiments of the present disclosure. These are reference spectra used to evaluate the dose response of aminobutyrate shown below.

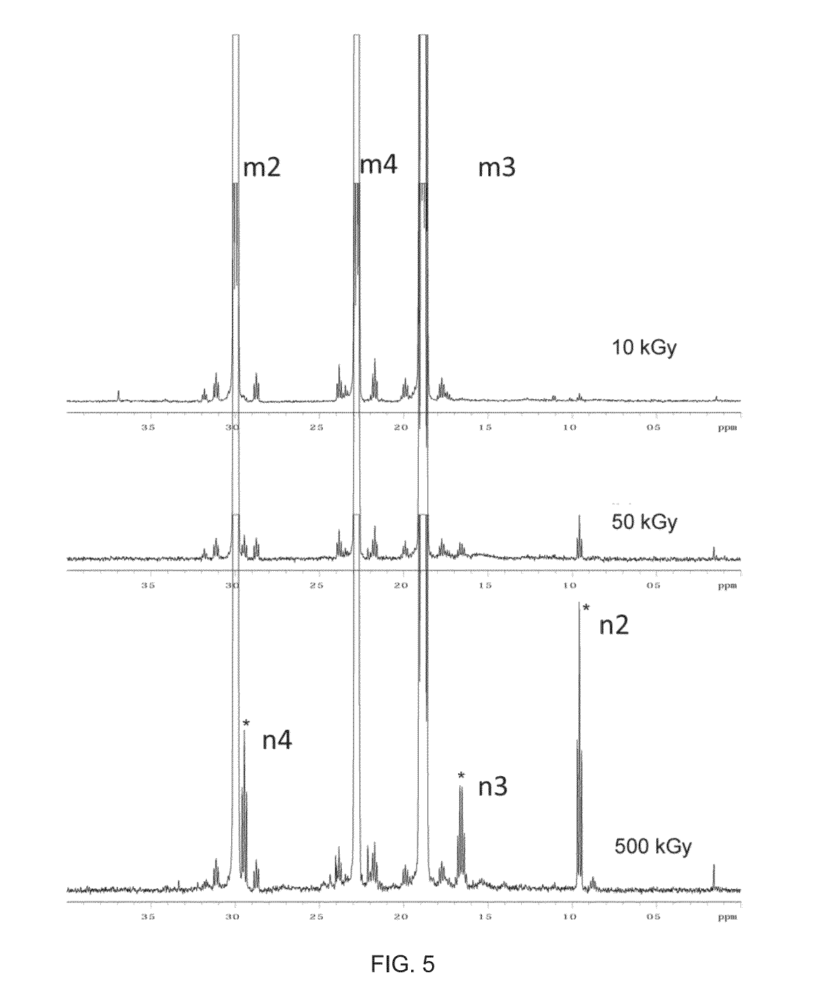

FIG. 5 shows an exemplary NMR Spectra of 4-aminobutyrate following 10 kGy, 50 kGy, 500 kGy doses, according to some embodiments of the present disclosure. Dry samples were irradiated in plastic centrifuge tubes at RT under air using X-rays generated by a tungsten tube operated at 70 kV. The results clearly show that the only dose-dependent peaks correspond to the formation of propylamine (see FIG. 4).

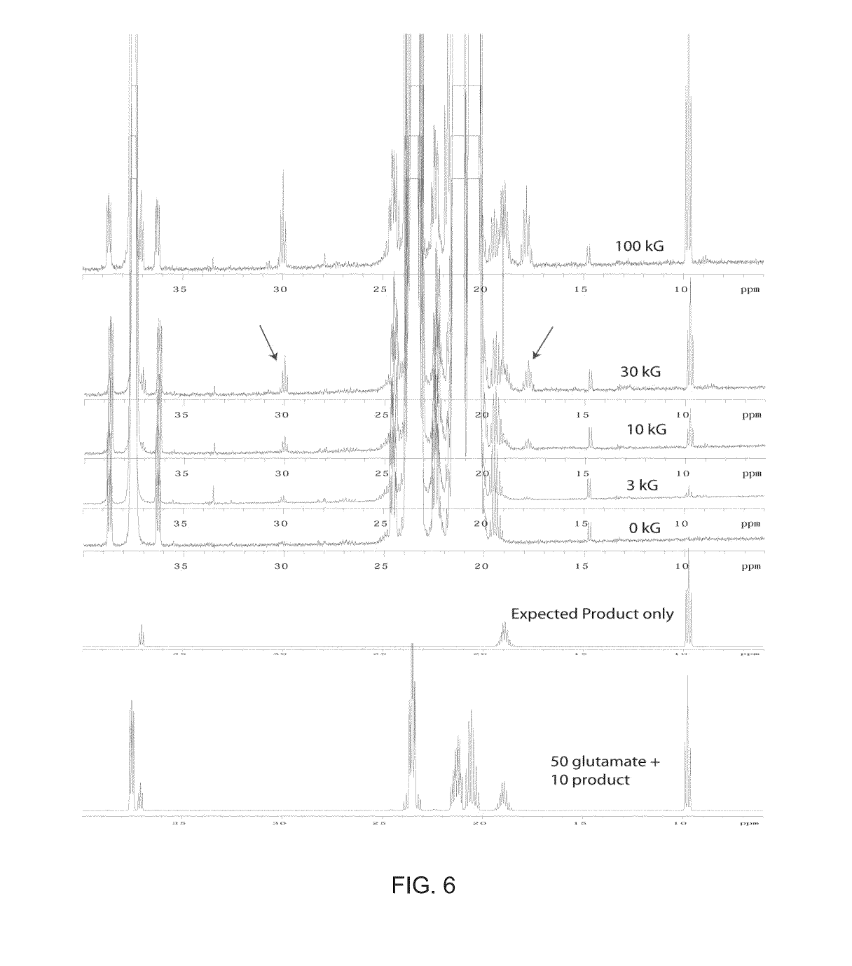

FIG. 6 shows an exemplary NMR Spectra of glutamate mixed with alpha-aminobutyric acid (bottom), alpha-aminobutyric acid (second from bottom), and glutamate following 0, 3 kGy, 10 kGy, 30 kGy and 100 kGy doses (top 5 spectra), according to some embodiments of the present disclosure. Dry samples were irradiated in plastic centrifuge tubes at RT under air using X-rays generated by a tungsten tube operated at 70 kV. The results clearly show that the expected product, alpha-aminobutyric acid, grows in a dose-dependant manner (along with other peaks that can be attributed to formation of gammaaminobutyric acid).

FIGS. 7A and 7B show an exemplary dose response of beta-alanine (top) and glutamate (bottom) as measured by integration of product peaks in NMR corresponding to ethylamine and alpha-aminobutyric acid, respectively, according to some embodiments of the present disclosure. The x-axis is dose (kGy) and the y-axis is normalized NMR peak area (unitless). The linear response of the products over this dose range is illustrative, and not limiting on the continued linearity beyond that range.

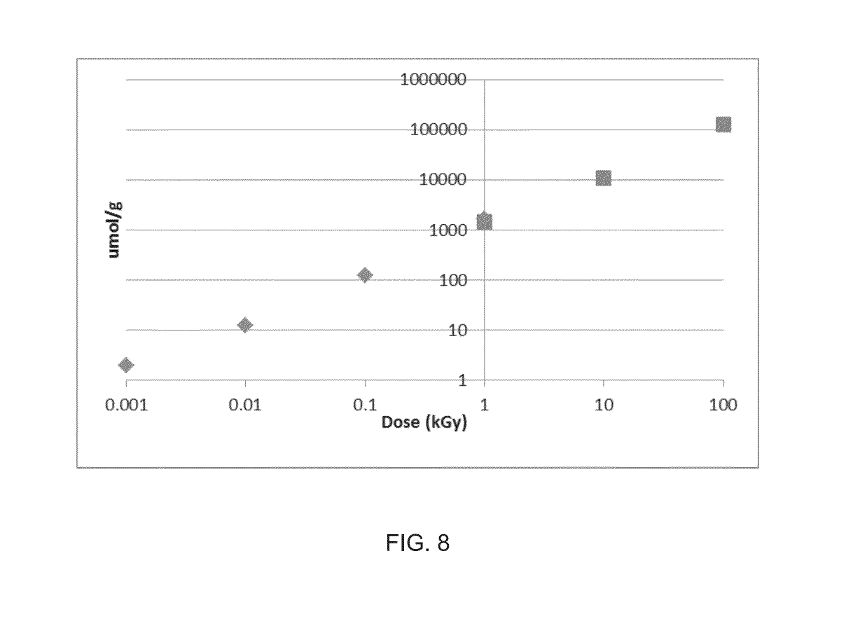

Additional preliminary evidence provided by liquid chromatography-mass spectrometry demonstrates that ex-vivo irradiation of glutamic acid produces alpha-aminobutyric acid, and that the dose response is again linear and is applicable to doses down to 1 Gy (FIGS. 7-8). This suggests that the technique is sensitive enough to be relevant as a biodosimeter.

FIG. 8 shows an exemplary dose response of glutamate as measured by liquid chromatography-mass spectrometry analysis and quantification of alpha-aminobutyric acid, according to some embodiments of the present disclosure. No signs of dose saturation are present, even down to 1 Gy.

According to some embodiments, based on results of a test procedure, a treatment plan or other corrective measures may be determined and executed. For example, upon determining an amount of radiation exposure, one or more treatment plans may be determined as suitable. For example, a lookup table may be provided with a pairing of radiation dosages with one or more treatment plans. By further example, a lookup table may be provided with a set of treatment plans correlated with one or more radiation dosages, wherein each of the treatment plans is provided with a probability of being a proper match with a respective radiation dosage amount or type. By further example, a lookup table may be provided with a range of treatment plans for selection by a user (e.g., a physician).

According to some embodiments, biologically relevant dosimetry can be obtained from a capillary of blood. For example, an assay investigation procedure may be performed to provide and analyze a sample from a patient.

For example, a pin prick to a capillary may yield 300 μL of blood. Based on a hemoglobin titer of 32-36 g/dl (e.g., about 0.34 g/ml or 0.34 mg/ml), 300 ml of blood would provide about 100 mg of hemoglobin. A radiation dosage of 1 Gy would provide a chemical yield of one-electron-oxidized glutamate (oeo-Glu) of about 1 nmol/J at 1 Gy (1 J/Kg). Under these circumstances, 100 μg of hemoglobin would provide 1×10⁻⁷ nmol of oeo-Glu. 0.1 fmol of oeo-Glu per Gy would be produced for each Glu in the hemoglobin. With 5 Glu per hemoglobin, this yields 0.5 fmol oeo-Gly/Gy. Therefore, it would be possible to detect as little as about 1 Gy of a dosage based on 300 μl of blood. The relationship is linear for values between about 1 mGy and about 10 MGy. Thus, greater dosages would also be detectable and measurable.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

A phrase such as “an aspect” does not imply that such aspect is essential to the subject technology or that such aspect applies to all configurations of the subject technology. A disclosure relating to an aspect may apply to all configurations, or one or more configurations. An aspect may provide one or more examples of the disclosure. A phrase such as “an aspect” may refer to one or more aspects and vice versa. A phrase such as “an embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples of the disclosure. A phrase such “an embodiment” may refer to one or more embodiments and vice versa. A phrase such as “a configuration” does not imply that such configuration is essential to the subject technology or that such configuration applies to all configurations of the subject technology. A disclosure relating to a configuration may apply to all configurations, or one or more configurations. A configuration may provide one or more examples of the disclosure. A phrase such as “a configuration” may refer to one or more configurations and vice versa.

As used herein, the phrase “at least one of” preceding a series of items, with the terms “and” or “or” to separate any of the items, modifies the list as a whole, rather than each member of the list (i.e., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

As used herein, the term “real time” shall be understood to mean the instantaneous moment of an event or condition, or the instantaneous moment of an event or condition plus short period of elapsed time used to make relevant measurements, optional computations, etc., and communicate the measurement, computation, or etc., wherein the state of an event or condition being measured is substantially the same as that of the instantaneous moment irrespective of the elapsed time interval. Used in this context “substantially the same” shall be understood to mean that the data for the event or condition remains useful for the purpose for which it is being gathered after the elapsed time period.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

While certain aspects and embodiments of the invention have been described, these have been presented by way of example only, and are not intended to limit the scope of the invention. Indeed, the novel methods and systems described herein may be embodied in a variety of other forms without departing from the spirit thereof. The accompanying claims and their equivalents are intended to cover such forms or modifications as would fall within the scope and spirit of the invention.

What is claimed is:

1. A method of determining an effect of ionizing radiation, comprising:
allowing a sample comprising a carboxylic acid to be exposed to an initially unknown amount of ionizing radiation, such that a single-electron oxidized product and carbon dioxide are produced from the carboxylic acid;
exposing the single-electron oxidized product to a radioprotectant, such that the single-electron oxidized product form an end product;
determining an amount of the end product; and
based on the amount of the end product, determining the amount of ionizing radiation.

2. The method of claim 1, wherein the sample comprises one or more of an amino acid, a dicarboxylic acid, a fatty acid, a sugar acid, a hydroxy acid, an acetic acid, a keto acid, a bile acid, a propionic acid, an aromatic acid, a carboxylic acid derivative, naphthoic acid, a nicotinic acid, a tricarboxylic acid, and a glucuronic acid.

3. The method of claim 1, wherein the sample comprises a beta amino acid.

4. The method of claim 1, wherein the sample comprises beta-alanine and the end product is ethylamine.

5. The method of claim 1, wherein the sample comprises gamma-aminobutyric acid and the end product is propylamine.

6. The method of claim 1, wherein the sample comprises glutamic acid and the end product is 4-aminobutyric acid and 2-aminobutyric acid.

7. The method of claim 1, wherein the radioprotectant comprises sulfur.

8. The method of claim 1, wherein the radioprotectant is ethanethiol.

9. The method of claim 1, whereby the determining is performed by one or more of gas chromatography, liquid chromatography, mass spectrometry, NMR spectroscopy, infrared spectroscopy.

10. A method of retrospectively determining an effect of ionizing radiation, comprising:
assaying a sample of tissue from a patient that has been exposed to ionizing radiation;
measuring an amount of a radiation product in the tissue, wherein the radiation product is a product of decarboxylation of an amino acid in the tissue in response to the ionizing radiation;
correlating the amount of the radiation product to an amount of ionizing radiation absorbed by the patient.

11. The method of claim 10, wherein measuring comprises extracting and hydrolyzing hemoglobin to produce a product.

12. The method of claim 11, wherein measuring further comprises analyzing the product by liquid chromatography coupled mass spectrometry

13. The method of claim 10, wherein the amino acid is beta-alanine and the radiation product is ethylamine.

14. The method of claim 10, wherein the amino acid is glutamic acid and the radiation product is alpha-aminobutyric acid.

15. The method of claim 10, wherein the tissue is blood.

16. The method of claim 10, wherein the ionizing radiation is provided as radiotherapy.

17. The method of claim 10, wherein the amount of ionizing radiation is between about 10 kGy and about 500 kGy.

18. A real-time dosimetry device, comprising:
a first chamber containing a reactant comprising a carboxylic acid configured to undergo loss of carbon dioxide to form an analyte, comprising a single-electron oxidized product, upon absorption of ionizing radiation;
a second chamber containing a detection reagent configured to emit a response in the presence of the analyte; and
a gas-permeable membrane separating the first chamber from the second chamber and configured to permit diffusion of the analyte.

19. The real-time dosimetry device of claim 18, wherein the second chamber contains a detection medium configured to emit a response in the presence of carbon dioxide.

20. The real-time dosimetry device of claim 18, wherein the reactant is beta-alanine and the analyte comprises ethylamine.
